(19) United States
(12) Patent Application Publication
(10) Pub. No.: US 2009/0062684 A1
(43) Pub. Date: Mar. 5, 2009

(54) APPARATUS AND METHOD FOR A GLOBAL MODEL OF HOLLOW INTERNAL ORGANS INCLUDING THE DETERMINATION OF CROSS-SECTIONAL AREAS AND VOLUME IN INTERNAL HOLLOW ORGANS AND WALL PROPERTIES

(75) Inventors: Hans Gregersen, Hornslet (DK); Flemming Gravesen, Aalborg (DK); Romulus Lontis, Aalborg O. (DK)

Correspondence Address: MERCHANT & GOULD PC, P.O. BOX 2903, MINNEAPOLIS, MN, 55402-0903, US

(73) Assignee: GLOBALREACH HOLDING APS, ARHUS N. (DK)

(21) Appl. No.: 11/910,097

(22) PCT Filed: Mar. 31, 2006
(86) PCT No.: PCT/DK2006/000188

§ 371 Date: Jun. 5, 2008

(30) Foreign Application Priority Data
Mar. 31, 2005 (DK) PA200500451
Jun. 10, 2005 (DK) PA200500852
Aug. 31, 2005 (DK) PA200501208

(51) Int. Cl. A61B 5/053

(52) U.S. Cl. 600/547

(57) ABSTRACT

The present invention relates generally to medical measurement systems for evaluation of organ function and understanding symptom and pain mechanisms. This model takes into account a number of factors such as volume and properties of the fluid and the surrounding tissue. Particular emphasis is on a multifunctional probe that can provide a number of measurements including volume of refluxate in the esophagus and to what level it extents. The preferred embodiments of the invention relate to methods and apparatus for measuring luminal cross-sectional areas of internal organs such as blood vessels, the gastrointestinal tract, the urogenital tract and other hollow visceral organs and the volume of the flow through the organ. It can also be used to determine conductivity of the fluid in the lumen and thereby it can determine the parallel conductance of the wall and geometric and mechanical properties of the organ wall.

### FIELD OF THE INVENTION

The present invention relates generally to medical measurement systems for evaluation of organ function and understanding symptom and pain mechanisms. This model takes into account a number of factors such as volume and properties of the fluid and the surrounding tissue. Particular emphasis is on a multifunctional probe that can provide a number of measurements including volume of refluxate in the esophagus and to what level it extents. The preferred embodiments of the invention relate to methods and apparatus for measuring luminal cross-sectional areas of internal organs such as blood vessels, the gastrointestinal tract, the urogenital tract and other hollow visceral organs and the volume of the flow through the organ. It can also be used to determine conductivity of the fluid in the lumen and thereby it can determine the parallel conductance of the wall and geometric and mechanical properties of the organ wall.

### BACKGROUND OF THE INVENTION

Visceral organs such as the gastrointestinal tract, the urinary tract and blood vessels all serve to transport luminal contents (fluids) from one end of the organ to the other end or to an absorption site. The esophagus, for example, transports swallowed material from the pharynx to the stomach. The esophagus has sphincters at both the proximal and distal entrance and the esophageal body in between. The area of the gastrointestinal tract between the esophagus and the stomach is known as the esophagogastric junction (EGJ). The mechanism which allows food to pass from the esophagus into the stomach and controls the amount of food and stomach acids from passing back up into the esophagus is know as the LES. Dysfunction of the LES can generally be related to two diseased states. Achalasia which is an uncommon primary esophageal motor disorder that is characterized by incomplete relaxation of the LES on swallowing and an absence of peristalsis of the esophageal body, and the much more common occurrence of gastro-esophageal reflux disease (GERD). GERD can occur when there is over exposure of the esophagus to acids refluxing back into the esophagus from the stomach. People suffering from GERD usually have heartburn and may have regurgitation and dysphagia. Recent figures indicate that up to 44% of the U.S. population suffers from GERD. GERD can result in damage to the mucosal lining of the esophagus, commonly referred to as esophagitis. Although the underlying cause of GERD is not exactly known it is related to two main patterns of sphincter dysfunction; an abnormally high rate of reflux episodes during transient LES relaxations and defective basal LES pressure. Patients with reflux symptoms may undergo endoscopy, manometric study of the esophagus and pH-metry. However, despite these methods is it unclear why a large number of the patients have reflux-like symptoms. Treatment of GERD can be pharmacological such as the use of PPI drugs, surgical such as the Nissen fundoplication or using newer endoscopic procedures such as the Gatekeeper procedure or stitching. PPI reduce the acid production in the stomach whereas the other techniques create an obstruction at the level of the lower esophagus/LES.

Diseases in visceral organs are often associated with symptoms and pain. The pain may develop due to several causes. e.g. in GERD it may be due to damage of the mucosa and penetration of acid or other substances that will affect nerve endings close to the mucosa. It would be of interest to develop a global model that considers a number of factors that can affect the system mediating symptoms and pain. For GERD this may include factors such as volume and acidity of the refluxate, the extent of reflux up in the esophagus, the mucosal barrier and its resistivity to penetration of protons and other substances, etc.

Diseases may affect the transport function of the organs by changing the luminal cross-sectional area, the peristalsis generated by muscle, or by changing the tissue components. Strictures in the gastrointestinal tract and coronary artery disease such as stenosis by an atherosclerotic plaque are examples. There is a need both for knowing the luminal cross-sectional area of the organ and the three-dimensional geometry of the lumen as well as it is important to know the structural, morphometric and mechanical properties of the organ wall. Impedance planimetry is a known technique for determining the lumen cross-sectional area during bag distension. Impedance measurements have also been used in terms of “intraluminal impedance” for detection of changes in resistivity during bolus passage. The great disadvantage is that the changes in resistivity (impedance cannot be converted to more useful measures such as lumen cross-sectional areas and information about the wall properties. Intraluminal ultrasound is another technique that can provide information about the lumen cross-section and wall thickness but its use it limited because it is a fairly expensive technique.

### SUMMARY OF THE INVENTION

The invention considers an overall model that can explain symptoms and pain in organs by taking into account a number of factors that affects the mucosa, receptors in the organ wall nerve pathways and the central control of the organ and sensation. In order to do this a complex mathematical model is needed and measurement tools must be implemented. In a first approximation the global model considers the organ to be a circular cylindrical tube, but the model may be refined if finer geometry can be measured, e.g. to take buckling of the organ into account. The model considers luminal factors such as the volume and the content of the fluid/material in the lumen, and how much of the mucosa/inner lining Is exposed the material/fluid. The model also Includes wall factors such as the penetration or perfusion constants through the mucosa/wall of chemicals/substances under suspicion for Inducing pain. The model can sum the factors together in different ways, for example it may be useful to integrate the acid load over the area of the mucosa taking the mucosal penetration of protons into account. With the esophagus as an example such substances may be protons, bile acids, drugs, etc. It is recognized in the model that damage to the mucosa will change the perfusion constants, and that these constants may vary throughout the esophagus. The constants can be derived from the literature or from simple experiments. One embodiment of this invention is a method to compute such constants. Another part of the invention is a method to determine the volume and the proximal extent of the refluxate in the esophagus or another organ.

The invention makes accurate measures of luminal cross-sectional areas of hollow internal organs. By measuring multiple cross-sectional areas it Is possible to determine the 3D geometry of the lumen and by using time delays and changes in cross-sectional areas as function of time It is possible to measure velocity of bolus transport and volume of the fluid passing the site of one or several sets of electrodes for the impedance measurements. More specifically in an embodiment the volume is determined by analyzing the data obtained from two adjacent cross-sectional area measurements and the time delay between them. Thus, the integrated cross-sectional area at one or both measurement sites multiplied by the distance between the two measurement sites and divided with the time between changes in the two channels provides the Information for the volume calculation. Other embodiments are based on different data analysis or measurements. Further, by measuring the conductivity of the fluid in the lumen it is possible to determine exact changes in the parallel conductance and thereby to obtain important information on the wall properties. The system may be combined with other techniques such as with probes containing stimulation modalities such as mechanical, chemical, electrical and thermal stimulations and sensors for recording of pressure, pH, axial force, etc. It may also consist of an array of ultrasound transducers on a probe in the lumen rather than using impedance measurements of the serial cross-sectional areas.

In one embodiment of the system and probe, the catheter contains a lumen where fluid can be sucked in or pass by. Alternative to having a small channel in the catheter for passage of fluid the catheter may contain 2 or more very closely spaced electrodes.

In one embodiment, impedance electrodes are supported at the tip of the catheter. These electrodes enable the immediate measurement of the cross-sectional area of the organ under study during advancement of the catheter. Error due to the loss of current in the wall of the organ and surrounding tissue can be corrected by Injection of two or more solutions of NaCl or other solutions with known conductivities, For the esophagus such errors may also be determined if the subject swallow fluids with known conductivity and/or with known volume. In this case the assumption is made that the size (cross-section) of the esophagus is the same during passage of the two or more boli. For other organs experiments can be set up In a similar fashion. The conductivity of the fluid can be measured by sucking the fluid into a channel or simple let it pass by a channel where electrodes and a constant current of constant voltage system can be used to determine the impedance. From the known size of the lumen it is possible to derive the conductivity of the fluid. Such an embodiment improves evaluation of cross-sectional area, flow and wall geometry and properties in other parts of the gastrointestinal tract or in organs like the cardiovascular system and the urinary tract

### BRIEF DESCRIPTION OF THE DRAWINGS

FIG. 1 is a schematic of one embodiment of the system showing a multifunctional catheter.

FIG. 2 shows a cross section of an 8-lumen tube or probe.

FIG. 3 shows a schematic of another embodiment of a multifunctional probe.

FIG. 4 shows additional schematic embodiments of multifunctional probes.

FIG. 5 shows calibration data for a four-electrode conductivity meter.

FIG. 6 shows a model for determination of parallel conductance.

FIG. 7 illustrates an example of a model of assessment of the bolus cross-sectional area.

FIG. 8 illustrates the relationship between the voltage, potential and the bolus radius.

FIG. 9 illustrates an example of differentiation of diameter curves for determination of geometric variables.

FIG. 10 illustrates a principle of stimulation and data acquisition.

FIG. 11 illustrates an example of a data acquisition set-up for impedance technique with parallel conductance in estimation of the esophagus lumen.

FIG. 12 illustrates the potential distribution.

### DETAILED DESCRIPTION OF EMBODIMENTS

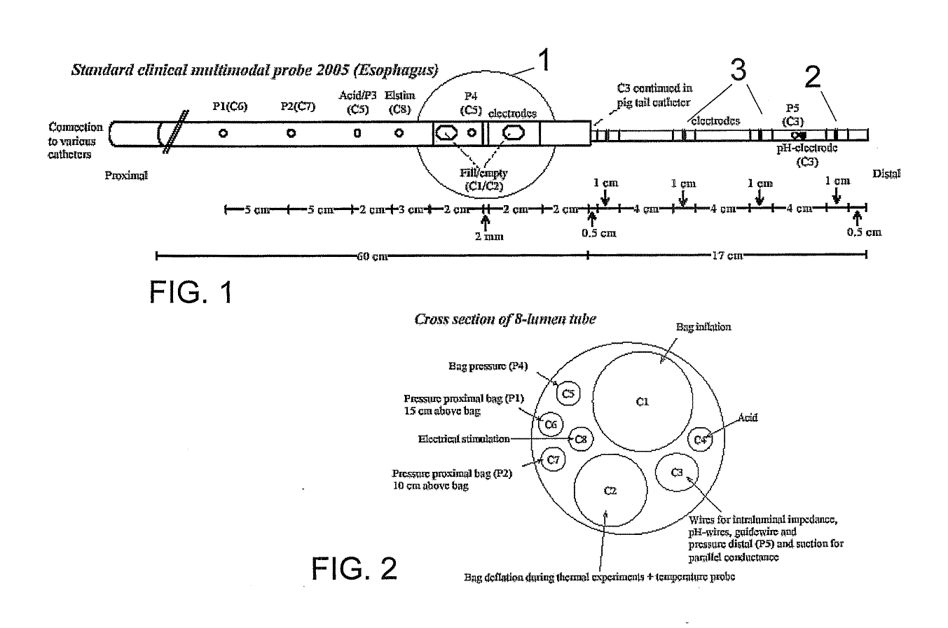

FIG. 1 is a schematic of one embodiment of the system showing a multifunctional probe, the probe also being referred to as a catheter, carrying Impedance measuring electrodes connected to the data acquisition equipment and excitation unit for the cross-sectional area measurement and a number of other measurements. In addition to the intraluminal impedance electrodes (e.g. 3), the catheter may contain a multifunctional system for mechanical, chemical, thermal and electrical stimulation of the organ and sensors such as axial force devices, pH sensors, conductivity sensor(s) 2, pressure transducers, etc. The impedance electrodes are preferably based on the four-electrode system, i.e. that two separate electrodes are used for excitation using a constant current and two inner electrodes are used for detection of potential differences. Alternative 2 electrodes may be used and electrodes may be used in common and combined in various ways. The probe contains pressure measuring sites (P1 to P5) distributed along the longitudinal axis of the probe. The pressure measuring sites is coupled to a lumen or channel running inside the probe (e.g. C6 and C7, see FIG. 2). Also a site for introducing acid (P3) is present on the probe, a site for electrical stimulation, i.e. an electrode (Elstim, C8). Also a balloon or bag 1 is attached to the probe, the balloon encircle electrodes as well as openings for filling and emptying of the balloon (C1/C2). The probe comprises a first thicker part, and a second thinner (pig tail) part.

FIG. 2 shows a cross section of an 8-lumen tube or probe. Uses of the lumen or channels are Indicated on the figure.

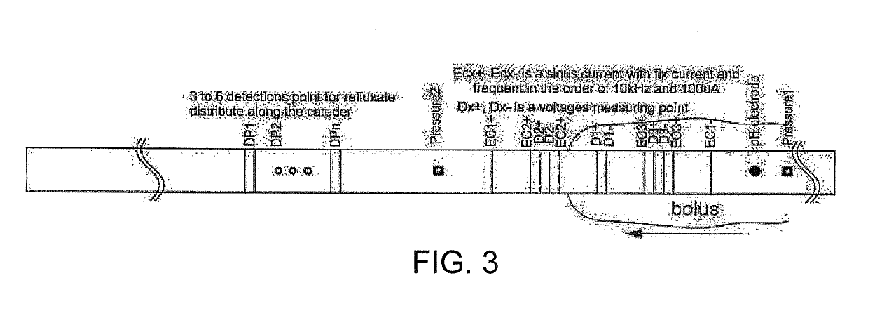

FIG. 3 shows a schematic of another embodiment of a multifunctional probe. In the figure pressure measuring sites, an pH electrode as well as configurations of impedance electrodes are illustrated.

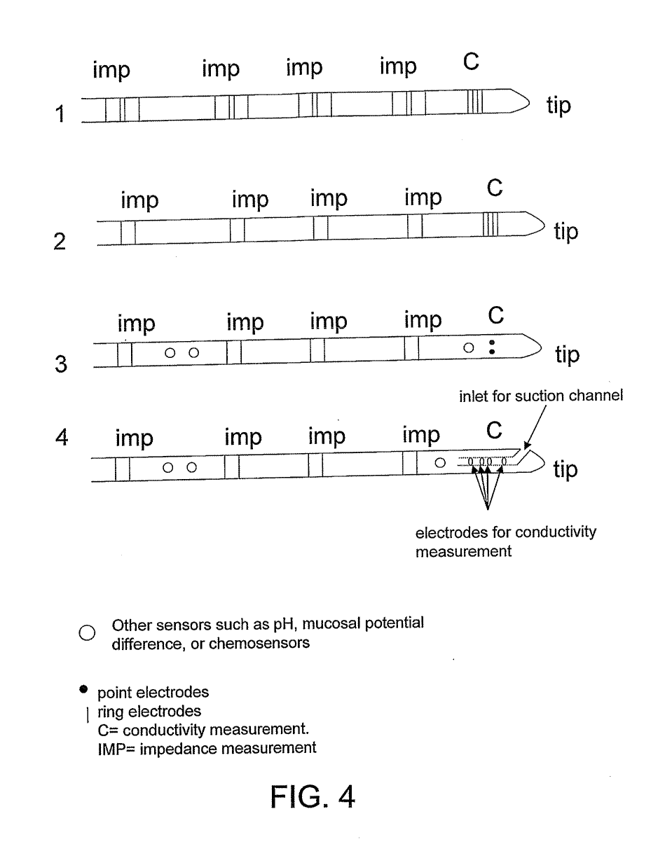

FIG. 4 illustrates three embodiments of the impedance-conductivity probe (#1 is 4-electrode principle, #2 is 2-electrode principle for impedance and 4-electrode for conductivity, #3 is 2-electrode for impedance and two point electrodes for conductivity, and #4 is with the electrodes (four electrodes in this embodiment) for conductivity measurements inside a suction channel. In #4 the suction channel exteriorizes again or It is connected to a suction device so continuous flow occurs in the channel. In #4 the conductivity can be determined because the diameter of the channel and the electrode spacings are known using Ohm's law. The embodiments can be combined in different ways and with different sensor types. The spacing of the conductivity electrodes, i.e. a first spacing may be in the order of 0.5 to 1 mm, so that a maximum extend may be up to 5 mm, whereas an extend of the impedance electrodes may be in the order of 1 to 2 cm. Thus, a first spacing of the electrodes of the conductivity sensor may be smaller than a second spacing of the electrodes of the Impedance sensor, e.g. by a factor 5 to 20.

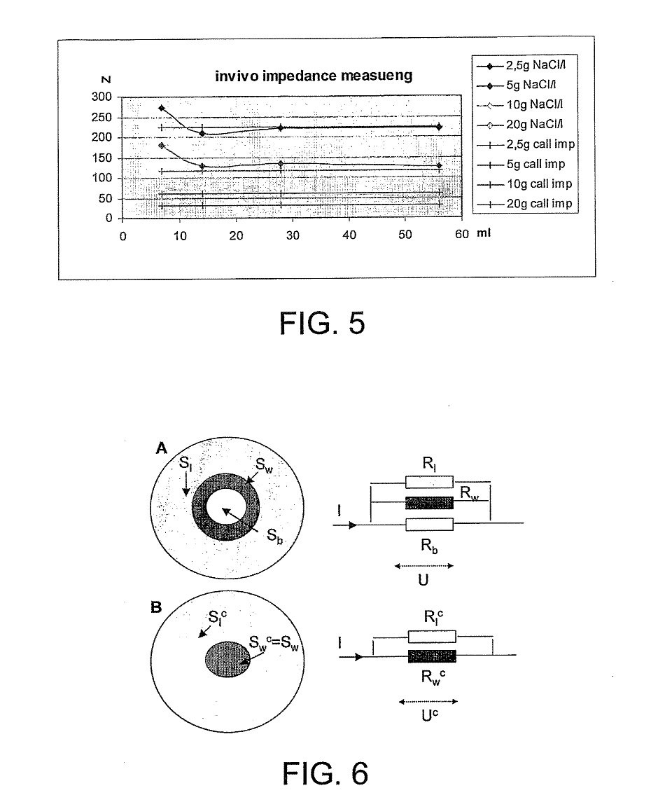

FIG. 5 shows calibration data for a four-electrode conductivity meter. The difference in baseline shows that the electrode system can be calibrated to measure conductivity.

The invention considers an overall model that can explain symptoms and pain in organs by taking into account a number of factors that affects the mucosa, receptors in the organ wall nerve pathways and the central control of the organ and sensation. In order to do this a complex mathematical model is needed and measurement tools must be implemented. In a first approximation the global model considers the organ to be a circular cylindrical tube but if finer geometry can be measured the model needs refinement, e.g. to take buckling of the organ Into account. The model considers luminal factors such as the volume and the content of the fluid/material in the lumen, and how much of the mucosa/inner lining is exposed the material/fluid. The model also includes wall factors such as the penetration or perfusion constants through the mucosa/wall of chemicals/substances under suspicion for inducing pain. The model can sum the factors together in different ways, for example it may be useful to integrate the acid load over the area of the mucosa taking the mucosal penetration of protons into account. With the esophagus as an example such substances may be protons, bile acids, drugs, etc. It is recognized in the model that damage to the mucosa will change the perfusion constants, and that these constants may vary throughout the esophagus. The constants can be derived from the literature or from simple experiments. One embodiment of this invention is a method to compute such constants. Another part of the invention is a method to determine the volume and the proximal extent of the refluxate in the esophagus or another organ.

The invention makes accurate measures of luminal cross-sectional areas of hollow internal organs. By measuring multiple cross-sectional areas it is possible to determine the 3D geometry of the lumen and by using time delays and changes in cross-sectional areas it is possible to measure velocity of bolus transport and volume of the fluid passing the site of one or several sets of electrodes for the impedance measurements. Further, by measuring the conductivity of the fluid in the lumen it is possible to determine exact changes in the parallel conductance and thereby to obtain important information on the wall properties. The system may be combined with other techniques such as with probes containing stimulation modalities such as mechanical, chemical, electrical and thermal stimulations and sensors for recording of pressure, pH, axial force, etc.

In one embodiment of the system and probe, the catheter contains a lumen where fluid can be sucked In or pass by. This lumen of known size will contain electrodes or sensors for determination of impedance, for example using a constant current and measuring the voltage will give the impedance and also the conductivity of the fluid. This will make possible determination of the conductivity of the fluid in the lumen, such as conductivity of the blood in a blood vessel, or the conductivity of the refluxate in the esophagus. The determination of the conductivity of the lumen fluid together with the changes in Impedance during passage of the fluid will allow detailed information of the system. It will be possible to convert the traditional measures of impedance to lumen cross-sectional areas and further information will be obtained about the wall geometry and electrical and elastic properties. Alternative to having a small channel in the catheter for passage of fluid the catheter may contain 2 or more very closely spaced electrodes. By passing a constant current or constant voltage between these electrodes, the highly nonlinear curve will help to determine the conductivity of the fluid. Assuming that the diameter of the lumen of the organ during the measurement is big compared to the distance between the electrodes, the curve form and especially the level of the asymptote provide information if the system is properly calibrated.

Embodiments of this invention overcome the problems associated with determination of the size (cross-sectional area) of luminal organs such as in the blood vessels, gastrointestinal tract, urethra and ureter. It is an improvement that the fluid conductivity is measured directly.

Assessment of Cross-Sectional Area in Esophagus Using Impedance-Metry with Estimation of the Parallel Conductance.

The set of measurements performed with the 2 pairs electrodes catheter in estimation of cross-sectional area must ensure the estimation of the parallel conductance. The simplest way to define the experimental protocol is to transfer the ‘problem’ into the lumped equivalent system that obeys the Ohm's law. The back transfer into the real system (the distributive system) can be performed through finite element analysis. In FIG. 6 the lung (or other surrounding tissue) (SI), esophagus wall (Sw) and the swallow or fluid bolus (Sb) domains are represented by circular cross-sections but all the computations below are still valid for any shape of the cross-section.

Principle: Since the estimation of the parallel conductance with the swallow substitution with two solutions of known conductivity is difficult to perform in practice the following approach is also proposed:
the parallel conductance (given by the wall and lung domains) modifies when inner diameter of the swallow domain (the one to be measured) varies. The extreme case is when the esophagus is closed.
a set of equations is used to ‘solve’ the parallel conductance only knowing the current injected, the voltage at the detection electrodes and the conductivity of the swallow using the geometric particularities involved between the two different cases (esophagus closed or of given diameter, FIG. 6A, B, left panels)
it is assumed that for any diameter of the swallow domain, the cross-surface of the wall has the same value i.e S_{w}^{c}=S_{w}
the equivalent resistance in the lumped model (FIG. 6 right panels) is R=d/(σS)⁽¹⁾, where d is the distance between the detection electrodes, σ is the conductivity and S the cross section for a given domain.

Accordingly, the following set of equations can be written:

$${R}_{L} \vert \vert {R}_{w} \vert \vert {R}_{S} = \frac{U}{I} \\ {R}_{L}^{c} \vert \vert {R}_{w}^{c} = \frac{{U}^{c}}{I} \qquad ( 2 )$$

and the surfaces in the two case are:

S_{w}^{c}=S_{w}

S_{L}^{c}=S_{L}+S_{S} (3)
results from (1), (2), (3);

$${σ}_{L} = {σ}_{S} - {I}^{*} \frac{d}{{S}_{S}} * ( \frac{1}{U} - \frac{1}{{U}^{c}} ) \qquad ( 4 )$$

Assuming that eq. 4 is valid for any cross-section of the swallow or bolus, if an experiment is performed when two solution of the same volume but of different conductivities σ_{S1} and σ_{S2} that produce the same cross-section of the esophagus S_{S1}=S_{S2} and the voltage measured U₁ and U₂ when a current I is injected than the conductivity of the lung σ_{L} can be computed as:

$${σ}_{L} = \frac{{σ}_{S 2} * ( \frac{1}{{U}_{1}} - \frac{1}{{U}^{c}} ) - {σ}_{S 1} * ( \frac{1}{{U}_{2}} - \frac{1}{{U}^{c}} )}{( \frac{1}{{U}_{1}} - \frac{1}{{U}_{2}} )} \qquad ( 5 )$$

and the dynamic evaluation of the cross-section of the swallow can be done with:

$${S}_{S} = I * \frac{d}{{σ}_{S} - {σ}_{L}} * ( \frac{1}{U} - \frac{1}{{U}^{c}} ) \qquad ( 6 )$$

Additional solutions. Equation (4) is a relation between two unknowns: the lung (or surrounding organ) conductivity σᵢ and the cross-sectional area S_{S}. The lung conductivity can be estimated and used in this way to simply calculate S_{S} from eq (4). This may introduce a systematic error. A calibration can be used to estimate a, using a balloon at the tip of the catheter and a solution of known conductivity to be injected thus the balloon blocks the solution passage into the stomach for short time, but enough to perform 2 (or more) measurements for 2 (or more) arbitrary cross-section (given by the balloon inflated) but not necessarily known as value. The system formed by multiple eq (4) can be used than to extrapolate the lung conductivity. Having this calibration done, the eq (4) can be used as reference from the lumped model for prediction of the bile domain cross-section. A finite element analysis can outline the nonlinearities between the lumped and the distributed models, with an estimation of the error involved. Rather than using one or several balloons to trap the fluid, it is possible to use fluids of known conductivities or to measure the conductivity using a small lumen with electrodes for electrical measurements. Hereby the bile conductivity can be measured and It is possible to estimate the cross-sectional area of the lumen fluid and of the wall through the determination of parallel conductance.

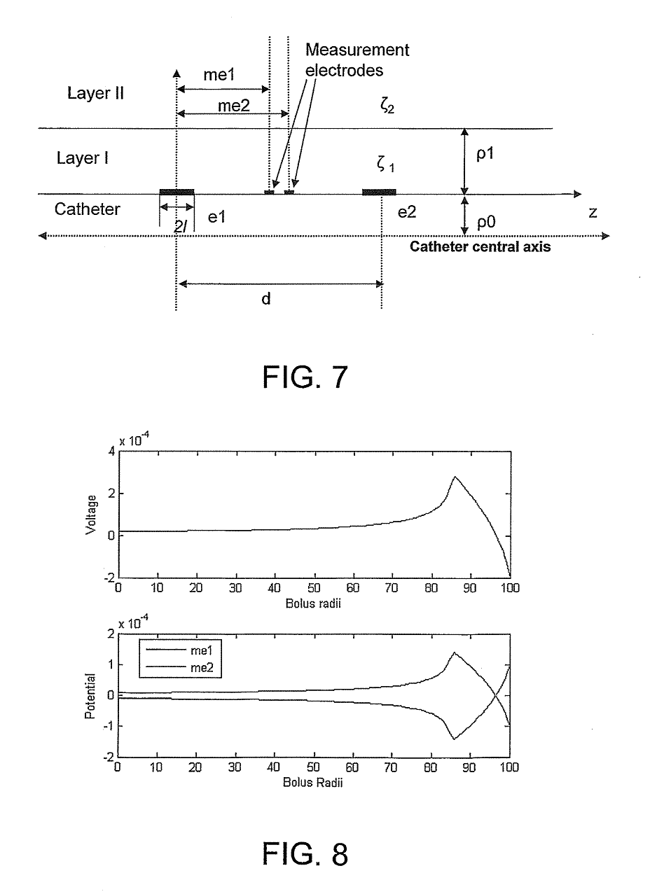

FIG. 7 illustrates an example of an assessment of the bolus cross-sectional area in oesophagus based on impedance measurements and the parallel conductivity. It shows a model of an infinite conductor with a catheter and two layers In cylindrical coordinates for simulation of the measurement electrodes and the volume conductor of the body around the catheter.

The bolus in the oesophagus or other internal organ is considered to be a cylindrical layer I with the conductivity ζ₁ (changes due to bolus contents) and with the thickness p1. The catheter will have a small size (radius p0) which can be subtracted from the p1 to correct for catheter size. The bolus encircles the catheter and lies in a homogeneous, infinitely extended volume conductor II with the conductivity ζ₂. The volume conductor II presents the conducting medium surrounding the bolus, e.g. the esophageal wall and other nearby tissues. The distances between an excitation electrode and the other excitation electrode, the nearby detection electrode and the far away detection electrode are named d, me1 and me2, respectively. The width of the excitation electrodes is 2I.

The potential function in layer I from two electrodes can be described as:

$${φ}_{1} = {\sum }_{e} \frac{{I}_{e} K}{{ζ}_{1}} [ ln \frac{{A}_{e} + \sqrt{{ρ}^{2} + {A}_{e}^{2}}}{{B}_{e} + \sqrt{{ρ}^{2} + {B}_{e}^{2}}} + \frac{{ζ}_{1} - {ζ}_{2}}{{ζ}_{1} + {ζ}_{2}} ln \frac{{A}_{e} + \sqrt{{( 2 {ρ}_{1} - ρ )}^{2} + {A}_{e}^{2}}}{{B}_{e} + \sqrt{{( 2 {ρ}_{1} + ρ )}^{2} + {B}_{e}^{2}}} ] \qquad ( 1 )$$

and for layer II

$${φ}_{II} = \frac{1}{4 π d ( {ζ}_{1} + {ζ}_{2} )} {\sum }_{e} {I}_{e} [ ln \frac{{A}_{e} + \sqrt{{ρ}^{2} + {A}_{e}^{2}}}{{B}_{e} + \sqrt{{ρ}^{2} + {B}_{e}^{2}}} ] \qquad ( 2 )$$

where
Aₑ=(d−z+l) for electrode 1
Aₑ=(z−l) for electrode 2
Bₑ=(d−z−l) for electrode 1
Bₑ=(z+l) for electrode 2

$$K = \frac{1}{8 π d}$$

The voltage between two measurement electrodes is:

V=φ_{I}|_{z}_{ₘₑ₂}_{,ρ=ρ}_{₀}−φₗ|_{z}_{_{=z}}_{ₘₑ₁}_{,ρ=ρp}_{₀}
d=20 mm,
l=0.4 mm
me1=9.5 mm

Hence, an example with me2=10.5 mm, then the relationship between the voltage,
ζ₁=15.5 e−3 s/cm
ζ₂=1.27 e−3 s/cm
η₀=1.5 mm
   potential and the bolus radius is as shown in FIG. 8.

The sensitivity of the system can be Improved in various ways such as by changing the distances between the electrodes

Embodiments also provide methods for registration of acute changes in wall conductance such as due to edema or acute damage to the tissue. Luminal cross-sectional area is measured by introducing from an exteriorly accessible opening or artificial opening of a hollow bodily system a catheter into the hollow system or targeted luminal organ. This catheter includes electrodes for accurate detection of organ luminal cross-sectional area and in a preferred embodiment also pressure measurements. The catheter can be inserted into the organs In various ways. Referring to FIG. 4 several variations of embodiments of the catheters can be made containing to a varying degree different electrodes, number and location of ports, and with or without multifunctional systems.

Medical imaging technologies may be used such as microsonometers, ultrasound and MR for calibration purposes or to measure parameters than will be useful in the analysis.

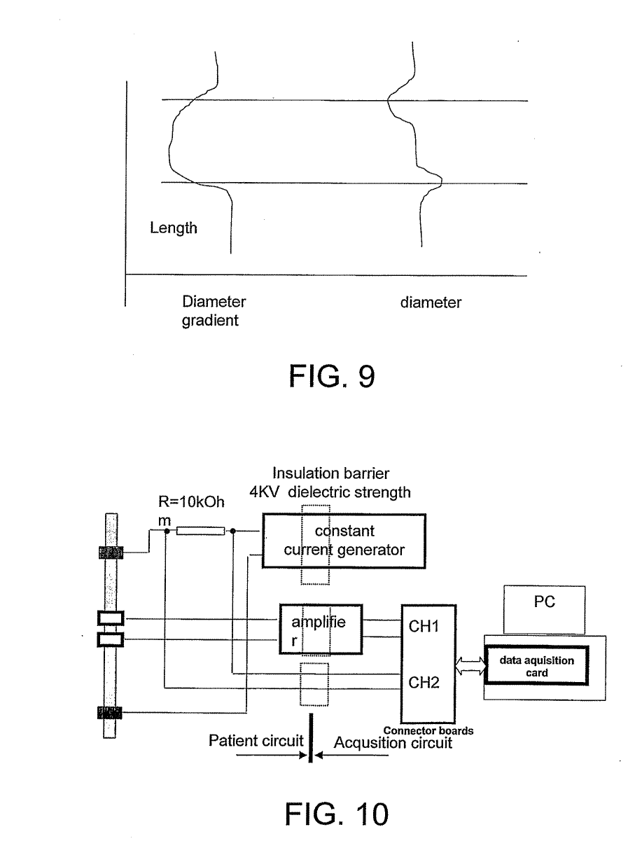

Other embodiments include various other sensors and applications. This includes a method for determination of tissue perfusion/ischemia using a bag where the temperature can be changed in a controlled fashion and changed. From the temperature versus time curve and the known volume, CSA and surface area of the balloon, heat exchange properties that depend on the mucosal perfusion can be derived. Other embodiments include an injection of fluid of known temperature into the stream and where a temperature sensor in the flow direction will provide curves that can be used to derive the flow in the organ. Other embodiments include placement of multiple impedance electrodes or other electrodes such as electrodes for transmucosal potential difference or pH in the circumference of the catheter or balloon in order to describe a circumferential variation in such parameters Other embodiments are
closely spaced electrodes for determination of fluid conductivity after proper calibration
including a device for viscosity measurements
integration of signals from impedances, conductances or imaging technologies to provide a 3D geometric and mechanical model
sensory assessment using evoked responses, VAS data, symptom reporting, functional brain imaging or referred pain assessment using PDA or solid or flexible monitors where the subject easily can draw the area of referred pain
where the frequency and amplitude of the induced current can be changed and where the number of electrodes to be used can be varied in terms of number and distances
use of a model where fluid and air can be differentiated and corrected for
using 2 or more ultrasound transducers or Doppler or m-mode ultrasound to generate the needed data for the model
implementation of muscle analysis such as generating pressure-CSA loops and tension-strain loops for individual contraction or preload-after load curves generated from P-CSA data or from imaging data
use of a miniature video camera or infrared alight or lasers to measure properties of the mucosal surface
to include electrodes on the catheter or bag for transluminal potential difference measurement
measurement of H+ disappearance in combination with other measurements as suggested in this description
use of hypertonic saline method or the use of change of electrical frequency to determine the parallel conductance and hence luminal and wall properties
One embodiment is an apparatus for determination of organ lumen and wall properties such as geometry, flow, luminal contents, functional measures, mass diffusion properties and sensory properties comprising a mathematical model that integrates lumen factors and wall factors
Another embodiment is an apparatus specifically for the esophagus with the purpose of understanding symptoms and esophageal disease in relation to resting conditions, swallows, reflux and other events.
Another embodiment is an apparatus where a catheter is used for stimulation and acquisition of data relevant to describe organ geometry and function
Another embodiment is a catheter and apparatus where the fluid viscosity and other fluid parameters such as pH, and electrolyte concentrations are measured.
Another embodiment is an apparatus where a number of electrodes are used to obtain data on lumen fluid conductivity and cross-sectional area and wall parallel conductance and area.
Another embodiment is an apparatus where sets of 2 electrodes are used to measure impedance
Another embodiment is an apparatus where 4 or more electrodes are used to measure one or more impedance signals to be used for the conductance analysis.
Another embodiment is an apparatus where the parallel wall conductance and thereby the lumen area and wall properties can be determined using injection of boli of known volume and conductivity, by changing conductance electrodes and combinations of electrodes, or varying the frequency or amplitude of the induced current.
Another embodiment is an apparatus where the conductivity of the fluid in the lumen can be determined inside a small lumen in the catheter equipped with electrodes or through a set of very closely spaced electrodes on the catheter.
Another embodiment is an apparatus where the cross-sectional areas and the change in cross-sectional area over time in adjacent channels is used to derive the volume of the fluid, the velocity of change, the extent where the fluid is present in the organ and other flow characteristics
Another embodiment Is an apparatus where corrections can be made for air In the volume of content.
A apparatus where volume and surface modeling is used the derive parameters of bolus and wall characteristics
Another embodiment is an apparatus where the organ geometry and mechanical properties can be determined during flow through the organ or contractile activity
Another embodiment is an apparatus where an array of electrodes are placed in the circumference of the catheter or in the circumference of the balloon/bag in order to obtain data on impedance, conductances, pH, transmucosal potential differences in order to characterize circumferential and local variations in parameters important for organ function and sensory function.
Another embodiment is an apparatus where ischemia in the tissue is evaluated using a bag where the temperature can be changed and measured and where mathematical equations can be used to derive a measure of tissue perfusion
Another embodiment is an apparatus where the preload-postload properties, pressure-CSA loops and tension-strain loops can be derived
Another embodiment is a probe and an apparatus where a miniature camera is placed close to the bag or tip in order to evaluate mucosal characteristics and damage to the tissue.
Another embodiment is an apparatus where the temperature of the flowing fluid can be changed by a controlled injection of fluid into the stream and where the temperature sensor further down the catheter measures and change in temperature from which a flow can be derived.
Another embodiment is an apparatus where the apparatus is used selectively to measure swallow induced activity and reflux episodes in the esophagus
Another embodiment is an apparatus that combines with sensory assessment in terms of symptom classification, VAS scores, evoked potential, functional imaging or referred pain assessment.
Another embodiment is an apparatus where the referred pain is measured using a PDA or solid or flexible digital board/plate from where the drawn areas are fed into a computer for online or offline recording of the area and its shape and location.
Another embodiment is an apparatus where the apparatus is used to measure in the cardiovascular system (vessels with changes In the wall such as atherosclerosis), the urogenital tract and other hollow internal organs
Another embodiment is an apparatus where impedances and lumen and wall conductances can be measured in the coronary arteries with the purpose of characterizing the wall and changes in the wall such as atherosclerotic plaques and their vulnerability
Another embodiment is an apparatus where imaging apparatus such as ultrasonography, MRI, scintigraphy etc. are used rather than impedance to obtain the needed data in the organ.
Another embodiment is an apparatus where several EUS transducers, Doppler ultrasound or m-mode ultrasonography is used to derive the data required in the model
Another embodiment is an apparatus where sensory responses are measured by means of evoked potentials, VAS scales, fMRI or corresponding apparatus
Another embodiment is an apparatus where mass diffusion in one of more dimensions Is part of the model for organ function evaluation
Another embodiment is an apparatus where air in the organ is accounted for and where gas and liquid can be distinguished by color coding
Another embodiment is an apparatus where the electrode spacings are varied with the purpose of providing the most useful data with respect to the air and air-liquid mix.
Another embodiment is an apparatus where correction for respiration and changes in lung conductivity is made
Another embodiment is an apparatus where saturation of the system is avoided by using appropriate liquids in the organ and scaling the measurement range of the equipment and probes electronically
Another embodiment is an apparatus where different electrode configurations are used such as multiple 2- and 4 electrode systems, several multielectrode systems with more than one set of excitation electrodes, or just one set of excitation electrodes and numerous sets of detection electrodes placing in between the excitation electrodes
Another embodiment is an apparatus where the part of the organ under study with respect to the luminal or surfaces is color coded with respect to geometry, pressures, pH, conductances or other measures
Another embodiment is an apparatus where the parallel conductance measures provides luminal CSAs that can be used for estimation of lumen, bolus and wall volumes by analyzing changes in CSA and other parameters between adjacent sites
Another embodiment is an apparatus where 2 or more closely spaced electrodes are used in organs including blood vessels to measure the conductance of the fluid in the organ
Another embodiment is an apparatus where the closely spaced electrodes are used after proper calibration in further analysis of organ function such as CSAs and volumes.
Another embodiment is an apparatus where parameters are computed such as bolus clearance, bolus presence time, total bolus transit time, cleared vs uncleared volume, length and diameter of volume distribution, closure diameter and pressure, bolus form, and opening velocity and shape.
Another embodiment is an apparatus where the parallel conductance measurements are combined with measurements of pressure, pH, bilitec or other chemical measurements
Another embodiment is an apparatus where the parallel conductance measurements are combined with swallow analysis and reflux analysis using predefined protocols.
Another embodiment is an apparatus where gas in the organ such as the esophagus is accounted for in the model by computing volume and distribution of gas in the organ, resistance against flow of gas and liquids
Another embodiment is an apparatus where different volumes of gas are swallowed in order to determine parameters such as the parallel conductance for the esophagus
Another embodiment is an apparatus where saliva is accounted for in the model and analysis and where the conductivity of saliva can be measured
Another embodiment is an apparatus where the CSA and volume data are used in further analysis of flow, volume loops, max flow-volume loops, isovolumteric pressure-flow (IVPF), flow-pressure loops, tension diagrams including active-passive tensions based on pharmacological modulation of organ function, mechanical parameters such as tension-strain, stress-strain relations, velocity curves and other muscle function and elasticity diagrams
Another embodiment is an apparatus where a perfusion test is done such as with water or saline of various conductivities or temperatures with the purpose of determining CSAs and volumes from analysis of variations in multiple impedance or pH measurements
Another embodiment is an apparatus where the changes in pH (velocity of change, magnitude and duration) along the esophagus after a reflux episode or swallows of known fluids are analyzed with respect to determining the volume of refluxed contents.
Another embodiment is an apparatus where the size of the catheter is corrected for in the analysis
Another embodiment is an apparatus where a resistance load parameter can be computed for the organ such as the lower esophageal sphincter since it is not a uniform tube, e.g. by integration along the length of the organ
Another embodiment is an apparatus where impedance and conductance measurements are used in characterizing body sphincters such as quantification of tLESPs in the lower esophageal sphincter
Another embodiment is an apparatus where the length of the section under study such as a sphincter or the tail of a bolus in the lumen is determined by analyzing mathematically the tracings such as differentiation of diameter curves along the sphincter in order to determine its length by defining local maxima and minima or other characteristics of the curve (FIG. 9)
Another embodiment is an apparatus where a gas (air) sensor is implemented in the probe or on a bag mounted on the probe
Another embodiment is an apparatus where such a sensor is an imaging device such as ultrasound with the purpose of detecting air with subsequent analysis of the data.

The signals are non-stationary, nonlinear and stochastic. To deal with non-stationary stochastic functions, we can use a number of methods, such as the Spectrogram, the Wavelet's analysis, the Wigner-Ville distribution, the Evolutionary Spectrum, Modal analysis, or preferably the intrinsic model function (IMF) method. The mean or peak-to-peak values can be systematically determined by the aforementioned signal analysis and used to compute the cross-sectional area (CSA).

We can measure the CSA at various time intervals and hence of different positions along the vessel to reconstruct the length of the vessel. This can then be displayed as a 2-D or 3-D Image of the vessel CSA and parallel conductance. This geometry can be determined of the lumen and of the wall and subsequently mechanical parameters can be derived such as stress-strain relations.

Electronic hardware and software used to interpret, display, calculate from and store data, will be configured to allow for different data collection configurations from the probe electrode array. Where suitable, signal multiplexing, synchronized array excitation and data sensing will be used to optimize the use of electrodes and signal lines. Electronic and physical switching could also be used to switch between electrode arrays or segments of electrode arrays.

For certain measurements the catheter will be fixed at the proximal end as well, i.e. is fixed to the nose or any other outer surface or organ. In the situation shown, where the catheter is introduced through the nose, fixation of the proximal end may take place in any suitable manner, perhaps by a clamp being clamped to the wing of the nose, to the nasal bone or to the bridge of the nose. Once introduced into the organ, the apparatus may be used for stimulating the sphincter of a person or an animal by mechanical stimulus. Alternatively, or in addition, the apparatus may be used for measuring a physical reaction of a person or an animal, when the bodily hollow system of the person or the animal is being subjected to a mechanical stimulus of the above-mentioned type. For cardiovascular use the catheter may be inserted through a femoral artery and advanced to the point of preferred measurement.

The catheter is provided with a number of channels running inside the catheter. Some of the channels are intended for passing stimulating means or measuring means from the proximal end of the catheter to a more distant end of the catheter.

Calculations of flow through the organ can also be made when the probe is in situ. The patient may perform both water and air swallows and the CSA and pressure are recorded. Using Newton's law of motion applied to force, rates for air and water in both the control and patient groups can be estimated using the following equation;

$$Q = Δ P \frac{{D}^{4}}{CVL}$$

Q=flow rate, ΔP=Pressure Difference, D=Diameter, C=Constant, V=Viscosity, L=length

Other suitable or possible ways of computing the flow rate may be applied under different geometric assumptions and depending on the design and function of the apparatus. Hence, using these equations together with the measurement of the volume passing a given point using the impedance technique, various unknowns such as the viscosity can be determined. Stiffness of the wall can estimated through wave analysis of the signals (small perturbations during the fluid transport).

### Example of Model Reconstruction Including Solid Model Re-Slicing and Surface Smoothing

The inner and outer contours for each cross-sectional image were Imported into and processed by MATLAB 6.5 software (The MathWorks Inc., Natick, Mass., United States). Hence, computation of the three-dimensional (3D) rectal surface was possible. This model was generated based on the transverse cross sectional images along the straight long axis of the stem part of rectum.

Since the distended rectum was deformed along a curved axis the alignment of data points along a curved axis was necessary to describe the rectal deformation at different distension volumes. By dividing the 3D model Into 30-39 equidistant segments along the curved center axis of the rectum the generation of a re-sliced solid 3-D model in any direction was possible.

The reconstructed surfaces also had some irregularities due to the discretization (artifacts and image analysis) of the images. The irregularities were reduced using a modified non-shrinking Gaussian smoothing method as outlined below.

### Computation of Geometric and Biomechanical Parameters

The surface area and the volume for both the Inner and outer contours were calculated based on the arc length and the cross sectional area as outlined below. The difference between the two volumes represents the volume of the rectal wall.

The circumferential strain was calculated based on the average of circumference in approximately the same 6 slices in both the stem and bending part of the rectum. The longitudinal strain was calculated in four different regions. In each region the longitudinal strain was based on the average length of approximately the same 10 longitudinal lines starting at the first and ending at the last slice. The strain ∈ was then calculated as the stretch ratio with the empty bag as reference length, ∈=l/lₒ.

The rectum has a complex 3D geometry. Since the surface is smooth and continuous, it was approximated locally by a biquadric surface patch. Hence, the principal curvatures, tension and stress were analyzed using a surface fitting method as outlined below. The peak tension was calculated as the highest tension in the entire rectal wall structure.

Based on the inner surface area A and the inner volume V of the 3D models a constructed bag length l was calculated based the on assumption of cylindrical shape l=A²/(V×4π). For evaluation of the present method an estimated radius r and tension T=p×r based on the assumption of both cylindrical r={square root over (V/(π×l)} and spherical r=\sqrt{3V/4π} shape were calculated.

### Surface Smoothing

The irregularities were removed using a modified non-shrinking Gaussian smoothing method, The relation between the position of the vertices before and after N iteration can be expressed as

X^{N}=((I−μK)(I−λK))^{N}X (A1)

where N was the number of iterations, λ and μ are two scale factors, I is the n_{V}×n_{V} identity matrix, K=I−W, W is the weight matrix and n_{V} is the number of the neighborhood of a vertex. In this study, λ=0.1 and μ=−0.101 to −0.103 were selected as the scale factors.

### Calculation of Geometric Characteristics

The approximate surface area and the volume were calculated from:

$$Sarea = {\sum }_{i = 1}^{n - 1} 0.25 * ( {arc}_{i} + {arc}_{i + 1} ) * ( {h}_{max} + {h}_{min} ) \\ Volume = {\sum }_{i = 1}^{n - 1} 0.25 * ( {area}_{i} + {area}_{i + 1} ) * ( {h}_{max} + {h}_{min} ) \qquad ( A2 )$$

where arcᵢ and areaᵢ is arc length and cross sectional area at a given cross section i, hₘₐₓ and hₘᵢₙ are the maximum and the minimum height between cross sections i and i+1 and n is the number of slices.

### Principal Curvature Computation

Since the surface is smooth and continuous, it can be approximated locally by a biquadric surface patch.

In this study, the local surface patch used is a tensor product B-spline surface as given by:

$$P ( u , v ) = {\sum }_{i = 0}^{2} {\sum }_{j = 0}^{2} {d}_{ij} {N}_{l}^{2} ( u ) {N}_{j}^{2} ( v ) \qquad ( A3 )$$

Each surface element consisted of 9 vertexes, three sequential points in the circumferential direction and three matching points (i.e., points originating from the same meridian) [16]. Thus, equation 3 can be expressed as:

$$X ( u , v ) = {\frac{1}{4} [ 1 u {u}^{2} ] [ \begin{matrix}1 & 1 & 0 \\ - 2 & 2 & 0 \\ 1 & - 2 & 1\end{matrix} ] [ \begin{matrix}{X}_{00} & {X}_{01} & {X}_{02} \\ {X}_{10} & {X}_{11} & {X}_{12} \\ {X}_{20} & {X}_{21} & {X}_{22}\end{matrix} ] [ \begin{matrix}1 & 1 & 0 \\ - 2 & 2 & 0 \\ 1 & - 2 & 1\end{matrix} ]}^{T} [ \begin{matrix}1 \\ v \\ {v}^{2}\end{matrix} ] ( u , v ∈ [ 0 , 1 ] ) \qquad ( A4 )$$

u, v are the coordinates in a local tangent plane coordinate system. The X matrix is the coordinates of the nine vertexes.

Then, the principle curvatures and principle directions for the central point can be calculated from the coefficient of the first fundamental form (E, F and G) and the second fundamental form (L, M and N) of the differential geometry as:

E=xᵤ² L=−xᵤNᵤ

F=xᵤxᵥ M=−xᵤNᵥ

G=xᵥ² N=−xᵥNᵥ (A5)

where

$$N = \frac{{x}_{u} \times {x}_{v}}{\vert {x}_{u} \times {x}_{v} \vert}$$

is the normal vector to the surface and the subscripts indicate partial differential (for example, xᵤ is the partial differential of x with respect to u). The principal curvatures k₁ and k₂ can be combined from the Gaussian curvature (K_{G}) and the Mean curvature (K_{M}):

$$\begin{matrix}{K}_{G} = {k}_{1} {k}_{2} = \frac{LN - {M}^{2}}{EG - {F}^{2}} & ( A6 ) \\ {K}_{M} = \frac{1}{2} ( {k}_{1} + {k}_{2} ) = \frac{1}{2} \frac{NE - 2 MF + LG}{EG - {F}^{2}} & ( A 7 )\end{matrix}$$

K_{G} is a particularly useful curvature parameter that indicates an elliptical surface (K_{G}>0), a parabolic surface (K_{G}=0) or a hyperbolic surface (K_{G<0}). K_{M} is in inverse proportion to the surface tension according to the Laplace's Law p=T*(k₁+k₂), where p denotes the transmural pressure acting on the surface, T is the surface tension which was assumed constant In every direction and k₁ and k₂ are the principal curvatures. The stress at a given surface point was calculated according to S=T/h_{wall}, where S is the stress, T is the tension and h_{wall} is the wall thickness at the point.

### Mass Diffusion Problem

It is of considerable Interest to include mass diffusion through the wall in a global organ model. For a one-dimensional model, three constants are needed, i.e. concentrations at the inner and outer surfaces and the diffusion coefficient that may change with temperature and pressure. The 1D model considers the radial direction only. The mass diffusion problem can better be described with 2D or 3D models which may require the use of a finite difference numerical method.

### A Data Acquisition Setup for Estimation of Parallel Conductance and Organ Lumen

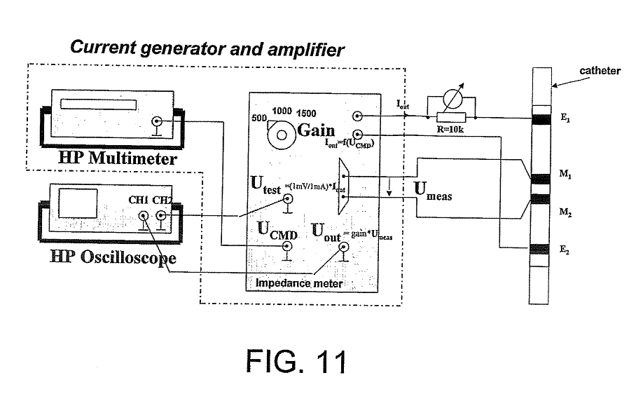

FIGS. 10 and 11 illustrates examples of a data acquisition set-up for the impedance technique with parallel conductance in estimation of the esophagus lumen.

### a) Principle of Stimulation and Data Acquisition (FIG. 10)

1. A constant current (10.100 μA, default 30 μA) is applied to the excitation electrodes (dark grey). A current detector (resistor R 10 kOhm in series with the excitation electrodes) is used to evaluate the current injected during experiment. The voltage across the resistor is applied to the Ch2 at the connector board (National Instruments BNC 2090).
2. The voltage from the detection electrodes (white) is amplified (usual gain is 500 to 1000 in order to amplify the voltage detected in the order of 1 mV up to 1V range . . . the data acquisition board is set-up for a −10 . . . 10 V range) and applied to the channel Ch1 at the connector board.
3. PC is equipped with National Instruments data acquisition board PCI 6024E. The acquisition is performed using MrKick and RunTime Engine 5.1 from LabView. The acquisition is performed continuously and the data is saved on the hard disk. To visualize the data from the hard disk must be used a Matlab routine (I can provide that).
4. 4. the circuit of voltage measurement from the resistor in series with the injection current circuit must have as well an Isolation barrier (usually implemented with an isolated amplifier with gain 1)
5. The catheter is inserted inside the oesophagus

### Remark

Both the current generator and the amplifier must provide an insulation with 4 kV dielectric strength if the set-up is used in human experiments. Additional must be used an isolator for the voltage detector from the resistor in series with the excitation electrodes (not used in the animal experiments).

Both current generator and amplifier were produced at SMI and they met the former safety standard requirements of 2 KV dielectric strength insulation, bellow is the set-up used previously.

### b) Practical Set-Up of Stimulation and Data Acquisition (FIG. 1)

FIG. 11 shows an example of assessment of cross-sectional area in esophagus using impedance-metry with estimation of the parallel conductance.

The current generator consists of HP 33120A (15 MHz Function/Arbitrary Waveform Generator) and the voltage to current transducer embedded in the Impedance meter IM001-05 produced at SMI in 1997 (it includes a 2 KV isolation barrier). The detected voltage is amplified through an isolated amplifier embedded as well in the impedance meter. A HP54600B with 2 channels can be used to monitor the different signal in the set-up.
Purpose: To evaluate the potential and current density distribution in a cylinder volume conductor (radius 30 mm, length 120 mm) composed by catheter (radius 2 mm), inner esophagus (radius 2.5, 5 and 10 mm, resp.) filled with bile (conductivity 1.4 S/m), esophagus wall (conductivity 0.53 S/m) and lung tissue (conductivity: inflated 0.09 S/m, deflated 0.23 S/m). Conductivity values are from the web site: http://niremf.ifac.cnr.it/tissprop/

The analysis performed must outline the current fraction that passes through different structures, the linearity of the potential distribution along the catheter and the current distribution along the normal above one of two central pair of detection electrodes. One pair of excitation electrodes was placed at z=±50 mm and another pair at z=±10 mm. Three pair of detection electrode, with distance between the electrodes of 4 mm were place around −20, 0 and 20 mm, respectively. The innermost detection electrodes from the pairs around −20 and 20 mm were used as well excitation electrodes. The voltage detected was analyzed only at the central detection electrodes pair. A current of 100 micro A was used.

Preliminary Conclusion The bile is very conductive (Due to high ions content . . . 1.4 S/m . . . close to the normal saline solution) and the esophagus wall does not represent barrier for the current (not proper isolation provided). Consequently the current passing outside the bile domain (i.e. the esophagus wall and the surrounding lung tissue) is relative high (Table 1) meaning that an estimation technique of the parallel conduction must be employed (unless the esophagus wall presents more isolating properties . . . like low conductance pleuras). The challenge is to obtain a uniform alteration of the bile conductivity for a high volume.

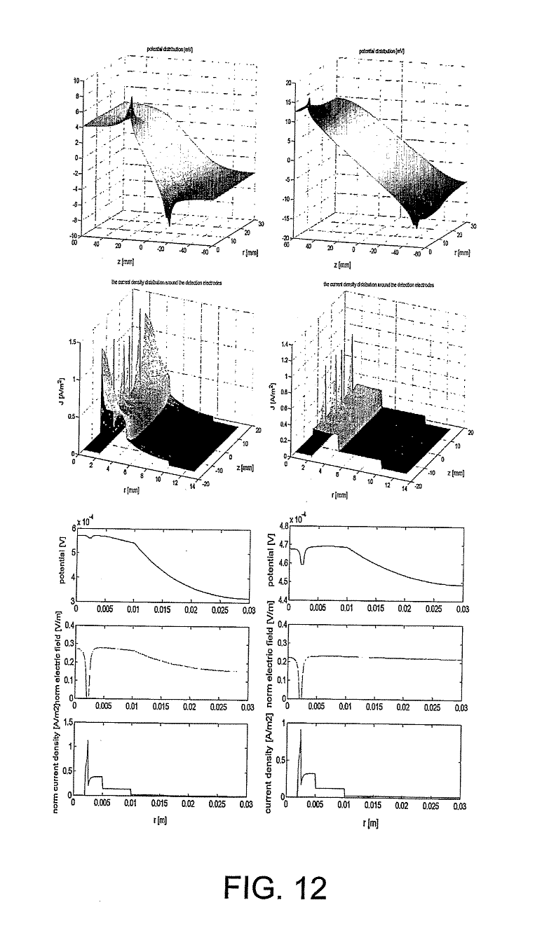

The current was injected though electrodes placed at different distances (table 1). The greater the distance the more current will flow outside the domain of interest (bile), but the linearity of the field over the detection electrodes is improved (see FIG. 12). There is an important nonlinear effect of the wall radius (table 1).

TABLE 1

| | | | | dₑₑ 40 | |
|---|---|---|---|---|---|
| | dₑₑ 100 | dₑₑ 40 | dₑₑ 40 mm | mm | dₑₑ 20 |
| | mm | mm | r_{cat} 2 mm | r_{cat} 2 mm | mm |
| | r_{cat} 2 mm | r_{cat} 2 mm | r_{wall} | r_{wall} | r_{cat} 2 mm |
| | r_{wall} 5 mm | r_{wall} 5 mm | 2.5 mm | 10 mm | r_{wall} 5 mm |
| Iₜₒₜₐₗ [μA] | 99.55 | 99.32 | 99.3 | 99.2 | 99.12 |
| I_{bile} [μA] | 21.05 | 25.58 | 4.2 | 55.37 | 31.82 |
| I_{wall} [μA] | 29.34 | 34.65 | 35.9 | 26.51 | 39.2 |
| U_{det} [mV] | 0.92 | 1.1 | 0.53 | 1.7 | 1.4 |
| Rₑₛₜᵢₘ [mm] | 10 | 9 | 13.1 | 7.3 | 8 |

1. An apparatus for determination of organ lumen and wall properties, the apparatus comprising:
an elongated probe having a distal and a proximal end,
at least one conductivity sensor, wherein one of the at least one conductivity sensor being positioned at a distal end of the probe
two or more impedance sensors distributed along the longitudinal axis of the probe.

2. An apparatus according to claim 1 further comprising a computing unit, and wherein the conductivity measured by the at least one conductivity sensor and the impedance measured by the two or more impedance sensor are inputted into a mathematical model that integrates lumen factors and wall factors, thereby calculating a cross-sectional area of a surrounding medium of the probe.

3. An apparatus according to claim 1, further comprising at least one sensor for measuring a fluid viscosity and other fluid parameters such as pH, and electrolyte concentrations.

4. An apparatus according to claim 1 where the conductivity sensor or the impedance sensor include a set of 2 electrodes.

5. An apparatus according to claim 1 where the conductivity sensor or the impedance sensor include a set of 4 or more electrodes.

6. An apparatus according to claim 1 wherein the probe further comprising a measurement lumen, and wherein the conductivity sensor is positioned in the measurement lumen.

7. An apparatus according to claim 1 wherein the conductivity sensor comprises a set of electrodes with a first spacing, and the impedance sensor comprise a set of electrodes with a second spacing, and wherein the first spacing is smaller than the second spacing.

8. An apparatus according to claim 2 wherein the mathematical model further includes volume and surface modeling to derive parameters of bolus and wall characteristics

9. An apparatus according to claim 1 wherein the conductivity sensor comprising an array of electrodes being positioned along a longitudinal axis or along the circumference of the probe.

10. An apparatus according to claim 1 further comprising a balloon positioned at the distal end and an array of electrodes, the array of electrodes being placed in the circumference of the balloon.

11. An apparatus according to claim 1 further comprising a miniature camera or sensors for mucosal potential difference being placed close to the tip of the probe in order to evaluate mucosal characteristics and damage to the tissue.

12-15. (canceled)

16. A method for determination of organ lumen and wall properties such as geometry, flow, luminal contents, functional measures, mass diffusion properties and sensory properties comprising a mathematical model that integrates lumen factors and wall factors.

17-19. (canceled)

20. A method according to claim 16 where a number of electrodes are used to obtain data on lumen fluid conductivity and cross-sectional area and wall parallel conductance and area.

21. A method according to claim 16 where sets of 2 electrodes are used to measure impedance.

22. A method according to claim 16 where 4 or more electrodes are used to measure one or more impedance signals to be used for the conductance analysis.

23. A method according to claim 16 where the parallel wall conductance and thereby the lumen area and wall properties can be determined using injection of boli of known volume and conductivity, by changing conductance electrodes and combinations of electrodes, or varying the frequency or amplitude of the induced current.

24. A method according to claim 16 where the conductivity of the fluid in the lumen can be determined inside a small lumen in the catheter equipped with electrodes or through a set of very closely spaced electrodes on the catheter.

25-26. (canceled)

27. A method according to claim 16 where volume and surface modeling is used the derive parameters of bolus and wall characteristics

28. (canceled)

29. A method claim 16 where an array of electrodes are placed in the circumference of the catheter or in the circumference of the balloon/bag in order to obtain data on impedance, conductances, pH, transmucosal potential differences in order to characterize circumferential and local variations in parameters important for organ function and sensory function.

30-41. (canceled)

42. A method according to claim 16 where mass diffusion in one of more dimensions is part of the model for organ function evaluation

43-44. (canceled)

45. A method according to claim 16 where correction for respiration and changes in lung conductivity is made

46. (canceled)

47. A method according to claim 16 where different electrode configurations are used such as multiple 2- and 4 electrode systems, several multielectrode systems with more than one set of excitation electrodes, or just one set of excitation electrodes and numerous sets of detection electrodes placing in between the excitation electrodes

48-52. (canceled)

53. A method according claim 16 where the parallel conductance measurements are combined with measurements of pressure, pH, bilitec or other chemical measurements

54-58. (canceled)

59. A method according to any of the claim 16 where a perfusion test is done such as with water or saline of various conductivities or temperatures with the purpose of determining CSAs and volumes from analysis of variations in multiple impedance or pH measurements.

60-63. (canceled)

64. A method according to claim 12 where the length of the section under study such as a sphincter or the tail of a bolus in the lumen is determined by analyzing mathematically the tracings such as differentiation of diameter curves along the sphincter in order to determine its length by defining local maxima and minima or other characteristics of the curve.

65-66. (canceled)
